(19) United States
(12) Patent Application Publication
(10) Pub. No.: US 2025/0099049 A1
(43) Pub. Date: Mar. 27, 2025

(54) SECURING AND COMPRESSING A BREAST IN MAMMOGRAPHY

(71) Applicant: Siemens Healthineers AG, Forchheim (DE)

(72) Inventors: Marcus RADICKE, Veitsbronn (DE); Carolin KUPILLAS, Erlangen (DE)

(73) Assignee: Siemens Healthineers AG, Forchheim (DE)

(21) Appl. No.: 18/895,628

(22) Filed: Sep. 25, 2024

(30) Foreign Application Priority Data
Sep. 27, 2023 (DE) 10 2023 209 451.7

(51) Int. Cl. A61B 6/04; A61B 6/50

(52) U.S. Cl. CPC A61B 6/0414; A61B 6/502

(57) ABSTRACT

A mammography compression device comprises a carrier unit having a first stop position and a second stop position. The mammography compression device also includes a breast support that is fixed at the first stop position of the carrier unit, and a compression paddle that is arranged at the second stop position of the carrier unit. The compression paddle is configured to pivot about a horizontal pivot axis, which runs through the second stop position, in the direction of the breast support.

### CROSS-REFERENCE TO RELATED APPLICATION(S)

The present application claims priority under 35 U.S.C. § 119 to German Patent Application No. 10 2023 209 451.7, filed Sep. 27, 2023, the entire contents of which is incorporated herein by reference.

### FIELD

One or more embodiments of the present invention relate to a mammography compression facility (also referred to as a mammography compression device). One or more embodiments of the present invention also relate to a method for automatically securing a breast of a female patient in a compression position for a mammogram. One or more embodiments of the present invention also refer to a breast compression method.

### BACKGROUND

Primarily rigid compression paddles are currently used in breast imaging. It is accordingly not possible to adjust them individually to the existing shape of the breast and they therefore lead to a suboptimal transfer of force to the breast. This results in an increased burden of pain for the female patient.

In addition, the positioning of the breast by the MTRA (Medical-Technical Radiology Assistant) is hampered. In order to secure the breast, the hand of the MTRA must necessarily rest on or adjacent to the breast and has to be withdrawn as soon as there is adequate effective securing of the breast by the paddle. The reason why compression paddles of different sizes are used is therefore because the MTRA can withdraw their hand under the paddle more easily/less painfully.

Various ideas exist for improving the compressing methods. These include curved but rigid compression paddles. Ideas for generating the compression by way of resilient materials or using, for example, an air cushion for compression are also known. However, none of the addressed methods have yet completely solved all the basic problems described and/or to some extent could not become established owing to associated drawbacks.

### SUMMARY

It is an object of one or more embodiments of the present invention to simplify and facilitate the positioning of the breast of a female patient before mammography.

At least this object is achieved by a mammography compression facility (also referred to as a mammography compression device), a method for automatically securing a breast of a female patient in a compression position for a mammogram, and a breast compression method as claimed.

The inventive mammography compression facility has a carrier unit. The carrier unit comprises a first end and a second end. Furthermore, the carrier unit comprises a first stop position and a second stop position. The first stop position is arranged closer towards the first end than the second stop position and the second stop position is arranged closer to the second end of the carrier unit.

The carrier unit is preferably also simultaneously part of a mammography facility and carries an X-ray source at a position in the direction of the second end or at the second end. In this sense the second stop position is positioned closer to the X-ray source than the first stop position.

A breast support is also part of the inventive mammography compression facility, and this is fixed at the first stop position of the carrier unit and extends transversely to the carrier unit.

Furthermore, the inventive mammography compression facility comprises a compression paddle which is arranged at the second stop position of the carrier unit. The compression paddle is embodied so it can pivot about a horizontal pivot axis, which runs through the second stop position, in the direction of the breast support. A compression paddle serves generally to compress the breast in order to improve the image quality in a mammogram.

The inventive mammography compression facility has, moreover, an adjusting mechanism for changing a height or a spacing of the second stop position relative to the first stop position. Conventionally, with proper positioning of the mammography compression facility, the second stop position is situated above the first stop position. However, a rotation of the mammography compression facility about 180° is also possible so the second stop position is then situated below the first stop position.

Finally, the inventive mammography compression facility also comprises a pivoting mechanism at the second stop position. The pivoting mechanism is configured to pivot the compression paddle in the direction of a part of a breast of a female patient located on an end of the breast support remote from the carrier unit. Furthermore, the pivoting mechanism is configured to exert a torque on the compression paddle in order to transfer a compression pressure of the compression paddle to the part of the breast of a female patient which is located on an end of the breast support remote from the carrier unit. The compression pressure is dimensioned such that it is sufficient to secure the breast. The pivoting mechanism is also configured to execute a pivoting-back of the compression paddle during a reduction of the spacing of the second stop position from the first stop position in order to thereby implement a planar securing of the breast. Preferably, but not necessarily, the compression paddle is pivoted back until in a horizontal position during the reduction of the spacing of the second stop position from the first stop position.

The mammography compression facility also comprises a keep-free unit for keeping a beam path for mapping the breast free of body tissue of the female patient which is situated in the surrounding area of the breast, with the breast of the female patient positioned as intended, preferably above the breast of the female patient.

The carrier unit is preferably embodied as a solid stand which is positioned on the floor. The “first end” or “lower end” should be taken to mean the end of the carrier unit which has contact with the floor when used as intended and is positioned closest to the floor. The “second end” or “upper end” should be taken to mean the part of the carrier unit furthest from the floor. Expressed in illustrative terms, the second stop position is positioned on the carrier unit closer to the X-ray source and the first stop position is arranged on the carrier unit further away from the X-ray source. The first stop position serves to secure the breast support of the inventive mammography compression facility. As a rule, the breast support is positioned height-wise as a function of the dimensions of a female patient, so the female patient can comfortably lay their breast on the breast support. The breast support then remains unchanged in its position during the positioning and securing of the breast.

By contrast, the compression paddle is inventively pivotably arranged at the second stop position of the carrier unit. The compression paddle is embodied so it can pivot about a horizontal pivot axis which runs through the second stop position. The pivotability of the compression paddle is preferably limited to the “downwards” direction, that is to say in the direction of the breast support which is situated “below” the compression paddle

In order to compress the breast in the horizontal position of the compression paddle, said adjusting mechanism is configured to change a spacing of the second stop position relative to the first stop position, in particular to reduce it. Expressed in illustrative terms, the compression paddle may therefore be pressed downwards against the breast of a female patient in order to compress the breast and thus obtain an improved image quality.

For pivoting the compression paddle, said pivoting mechanism is embodied at the second stop position. A torque is exerted on the compression paddle by the pivoting mechanism. This torque is preferably mechanically transferred to the compression paddle by an actuator. The compression pressure of the compression paddle generated thereby is dimensioned such that it is sufficient to secure the breast of a female patient. Owing to the diagonal orientation of the compression paddle when securing the breast it is considerably easier for an MTRA to withdraw a hand from under the compression paddle since there is more space under the compression paddle in this position than when the compression paddle rests horizontally on the breast.

If the compression paddle at the second stop position is now lowered further downwards, that is to say in the direction of the breast support, the position of the compression paddle thus changes from a diagonal position to more of a horizontal position or completely horizontal position of the compression paddle. Therefore, the compression paddle is pivoted back into a less diagonal and preferably horizontal position during a reduction of the spacing of the second stop position from the first stop position. Once the compression paddle has reached the target position, preferably a horizontal position, the process of actually compressing the breast can begin immediately before the start of imaging. The second stop position preferably has a mechanical blocking device in order to prevent further pivoting of the compression paddle “upwards” beyond the target position, preferably a horizontal position or in the direction of the second end of the carrier unit and thus with further lowering of the compression paddle or as the compression paddle approaches the first stop position, to increase the pressure exerted on the breast and to press the breast in the target position, preferably a horizontal position, of the compression paddle in a more or less flat manner.

The inventive mammography compression facility preferably comprises a control facility for actuating the movable elements of the mammography compression facility, in particular for actuating the pivoting mechanism and the adjusting mechanism of the inventive mammography compression facility. Individual processes of securing and compressing can be carried out partially or completely automatically with this control facility.

In the inventive method for automatically securing a breast of a female patient in a compression position for a mammogram, the breast is positioned on a breast support. The breast support is fixed at a first stop position of a carrier unit. The carrier unit comprises a first end and a second end.

The breast support extends transversely to the carrier unit. The carrier unit also comprises a second stop position which is arranged closer to the second end than the first stop position.

In the inventive method, a compression paddle, which is arranged at the second stop position, is pivoted about a horizontal pivot axis, which runs through the second stop position, in the direction of the breast support. The compression paddle is pivoted in the direction of a part of a breast of a female patient located on an end of the breast support remote from the carrier unit by a pivoting mechanism at the second stop position, and a torque is exerted on the compression paddle in order to transfer a compression pressure of the compression paddle to a part of the breast of a female patient. This part of the breast rests on the end of the breast support remote from the carrier unit. The compression pressure and therewith the torque are selected such that they are sufficient to secure the breast.

Furthermore, a spacing of the second stop position relative to the first stop position is reduced by an adjusting mechanism. In this context, the compression paddle is pivoted back into a less diagonal position, preferably a horizontal position, than when securing the breast in order to thereby implement planar securing of the breast. Furthermore, a beam path for mapping the breast is kept free of body tissue of the female patient which is situated in the surrounding area of the breast, preferably above the breast of the female patient. The inventive method for automatically securing a breast of a female patient in a compression position for a mammogram shares the advantages of the inventive mammography compression facility.

In the inventive breast compression method, firstly the inventive method for automatically securing a breast of a female patient in a compression position for a mammogram is carried out. Once the breast is secured it is compressed in a planar manner in the position reached during the securing process, preferably a horizontal position, of the compression paddle as the next step. Once a predetermined compression of the breast is achieved, imaging of the breast is then carried out and a mammogram of the breast of the female patient is generated. The inventive breast compression method shares the advantages of the inventive method for automatically securing a breast of a female patient in a compression position for a mammogram.

One or more embodiments of the present invention can be implemented, in particular, in the form of a computer unit with suitable software. The computer unit can have, for example, one or more cooperating microprocessor(s) or the like for this purpose. In particular, it can be implemented in the computer unit in the form of suitable software program parts. An implementation largely in terms of software has the advantage that even previously used computer units can be easily retrofitted by way of a software or firmware update in order to work inventively. In this regard the object is also achieved by a corresponding computer program product with a computer program which can be loaded directly into a storage facility of a computer unit, preferably a control facility of a mammography system, with program segments in order to execute all steps for controlling the inventive method for automatically securing s breast of a female patient in a compression position for a mammogram and for controlling the inventive breast compression method when the program is executed in the computer unit. Apart from the computer program such a computer program product can optionally comprise additional elements, such as documentation and/or additional components, also hardware components, such as hardware keys (dongles, etc.) in order to use the software.

A computer-readable medium, for example a memory stick, a hard disk or another transportable or permanently installed data carrier can serve for transporting the computer unit and/or for storage on or in the computer unit, on which medium the program segments of the computer program, which can be read-in and executed by a computer unit, are stored.

Further, particularly advantageous embodiments and developments of the present invention can be found in the dependent claims and the following description, with it being possible to also develop the claims of one category of claims analogously to the claims and descriptive parts relating to a different category of claims and, in particular, to also combine individual features of different exemplary embodiments or variants to form new exemplary embodiments or variants.

It is preferred that in one embodiment of the inventive mammography compression facility, the compression paddle has a pressing body, wherein the pressing body has a flexible material. The pressing body forms the part of the compression paddle which is pressed against the breast of a female patient in order to compress it. Due to its flexibility the pressing body can adapt to the shape of the breast and thus bring about a more uniform pressure distribution during the compressing procedure in comparison with a rigid conventional compression paddle. On the other hand, the material of the pressing body should have sufficient strength to enable a minimum pressure for securing the breast. It is also preferred that it is possible to clean the material of the pressing body.

In one variant of the inventive mammography compression facility, the material of the pressing body is a single-use material. Advantageously, the pressing body can be replaced before each examination for hygiene reasons. Alternatively, the pressing body can also have a film which likewise serves as a form of hygiene protection for the female patient and can likewise be easily replaced.

Alternatively, the pressing body of the inventive mammography compression facility can also comprise a strong rigid material, in particular polyethylene terephthalate or acrylic glass, in order to transfer a strong pressure to the breast of a female patient.

In a preferred embodiment of the inventive mammography compression facility, the flexible material comprises a gauze material. Gauze is flexible but also very robust and may be stabilized by a tensioning procedure in order to achieve the necessary strength in the pressing procedure of the breast. The gauze clings to the breast of the female patient and thus allows a uniform pressure distribution on the breast. Advantageously, patient comfort is also increased thereby.

It is likewise preferred that the compression paddle of the mammography compression facility has a frame in which the pressing body is mounted. The frame of the compression paddle is preferably arranged at the periphery of the compression paddle and provides fixing points for the pressing body.

In one variant of the inventive mammography compression facility, the frame is formed laterally and on the side facing the carrier unit or the side of the compression paddle remote from the female patient. Expressed in illustrative terms, the frame has a U-shaped form to which the pressing body can be fixed. The frame opens in the direction of the patient so a female patient does not come into contact with the frame but only with the pressing body.

It is preferred that in one embodiment of the inventive mammography compression facility, the pivoting mechanism comprises a hinge, preferably a friction hinge. The friction hinge has a restoring force which is sufficient to generate a pressure that acts on the breast of a female patient which is sufficient to secure the breast of the female patient. Initially, the breast is therefore not compressed more, but merely secured.

The pose of the compression paddle and the selected pressure for securing the breast can also be ascertained on the basis of algorithms, with an optimum pressure and/or angle of the compression paddle being ascertained on the basis of breast parameters.

In a preferred embodiment of the inventive mammography compression facility, the pivoting mechanism has a mechanical blocking device, preferably a stop, for limiting a pivoting range of the compression paddle to a lower quadrant. The stop forms a positive fit with the compression paddle when the compression paddle comes into contact with the stop. The stop is preferably arranged opposite the second stop position or the pivoting mechanism, preferably a hinge of the pivoting mechanism. Advantageously, the position of the compression paddle is held in a horizontal position against the breast of a female patient during a pressing procedure of the compression paddle, so the compression of the breast is uniformly increased as the compression paddle approaches the breast.

In a preferred embodiment of the inventive mammography compression facility, the keep-free unit of the inventive mammography compression facility has a displaceable plate which is embodied so it can be transversely displaced relative to the horizontal pivot axis of the compression paddle up to an end of the compression paddle remote from the carrier unit and is transversely oriented relative to the compression paddle. The displaceable plate solves the problem of body tissue situated in the surroundings of the breast, in particular the tissue and the skin located above the breast in the decollete region, which is also referred to as the skin of the upper chest, tending to protrude beyond the edge of the breast during a mammogram, and potentially generating X-ray artifacts. This is more frequently the case in particular with obese patients but equally with older female patients. The skin of the upper chest is accordingly inventively squeezed by the displaceable plate. This displaceable plate is not in its final position on initial contact between the compression paddle and the breast, rather it is displaced in the direction of the carrier unit so as to be remote from the breast. As soon as the horizontal position of the compression paddle is reached, the displaceable plate is automatically or manually pushed into its final position on the breast wall and is thus pressed horizontally against the skin of the upper chest. Advantageously, the skin of the upper chest can be kept out of the beam path, so the exposure of the female patient to radiation is minimized and image artifacts, which originate from the skin of the upper chest that is held in the image region, can be prevented.

It is therefore likewise preferred that in one embodiment of the mammography compression facility, the compression paddle has a carriage guide running transversely to the horizontal pivot axis of the compression paddle and the displaceable plate has a carriage which engages in the carriage guide in such a way that the displaceable plate can be displaced transversely to the horizontal pivot axis of the compression paddle. Advantageously, the plate can be displaced by this mechanism to a different position as required. In particular, the displaceable plate is only displaced to the edge of the breast when the compression plate is in a target position, preferably a horizontal position.

The carriage guide can be embodied, in particular, as a dovetail guide in order to guide the movement of the displaceable plate. Advantageously, the carriage of the displaceable plate is prevented from being lifted out in the normal direction of the sliding plate by the positive fit of the carriage with the dovetail guide.

In one variant of the inventive mammography compression facility, the mammography compression facility has a securing mechanism for securing the displaceable plate at a final position at the end of the compression paddle remote from the carrier unit. Advantageously, this securing mechanism ensures that the displaceable plate remains in a final position at the edge of the breast of a female patient during a scanning procedure.

Preferably, a latching process takes place for this purpose, and this can take place manually or automatically. It is thus also preferred that in one embodiment of the inventive mammography compression facility the securing mechanism of the inventive mammography compression facility comprises a latching mechanism. Advantageously, the position of the displaceable plate remains substantially unchanged relative to the compression plate and the skin of the upper chest during a compression procedure of the breast, so a displacement of the skin of the upper chest from the radiation field of the mammography system is ensured.

The displaceable plate can also have a stabilizing effect for the pressing body, in particular the gauze, so the main compression procedure begins only after reaching the final position of the displaceable plate since previously this stabilizing effect still did not occur on the pressing body.

Preferably, the displaceable plate has polyethylene terephthalate or carbon fiber-reinforced plastic as the material. Advantageously, these materials have sufficient strength to squeeze skin of the upper chest and optionally also to stabilize the pressing body.

### BRIEF DESCRIPTION OF THE DRAWINGS

The present invention will be explained once again in more detail below on the basis of exemplary embodiments with reference to the accompanying figures. In the drawings:

FIG. 1 shows a schematic side view of a mammography compression facility according to one exemplary embodiment of the present invention with a compression paddle in a diagonal position,

FIG. 2 shows a schematic side view of the mammography compression facility illustrated in FIG. 1, with the compression paddle being horizontally oriented,

FIG. 3 shows a schematic plan view of a compression paddle of a mammography compression facility according to one exemplary embodiment with a pivoting mechanism with two hinges,

FIG. 4 shows a schematic plan view of a compression paddle of a mammography compression facility according to one exemplary embodiment with a pivoting mechanism with just a single hinge,

FIG. 5 shows a flowchart, which illustrates a breast compression method, which also comprises a method for automatically securing a breast of a female patient in a compression position for a mammogram according to one exemplary embodiment of the present invention.

### DETAILED DESCRIPTION

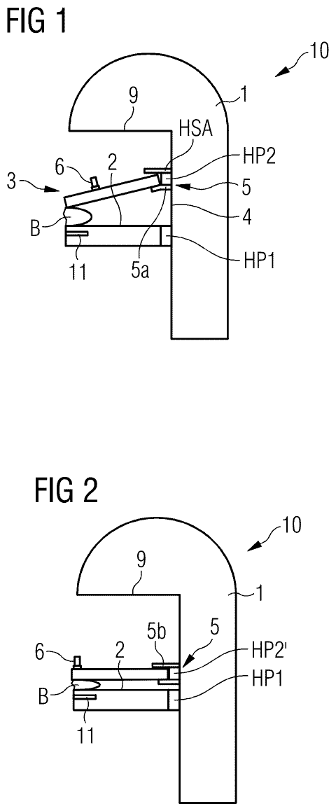

FIG. 1 shows a schematic side view of a mammography compression facility 10 according to one exemplary embodiment of the present invention with a compression paddle in a diagonal position. The mammography compression facility 10 has a carrier unit 1. A combination of an X-ray source 9 and an X-ray detector 11 is arranged on the carrier unit 1. The X-ray source 9 is situated at an upper end of the carrier unit 1 and the X-ray detector 9 is situated below a breast support 2 which is likewise part of the mammography compression facility 10. The breast support is situated at a first stop position HP1 which is approximately arranged at breast height of an average female patient.

A compression paddle 3, which is likewise part of the mammography compression facility 10, is arranged at a second stop position HP2 which is situated above the first stop position HP1. The compression paddle 3 is embodied so it can pivot about a horizontal pivot axis HSA, which runs through the second stop position, in the direction of the breast support 3. FIG. 1 represents a diagonal position of the compression paddle 3. In this position an end of the compression paddle 3 remote from the carrier unit 1 is in contact with the breast B of a female patient. A pivoting mechanism 5, which comprises a carrier-side hinge 5a on which the compression paddle 3 is suspended on the carrier side, is also part of the mammography compression facility 10. When the compression paddle 3 is pivoted from the horizontal position (not shown) into the diagonal position, the pivoting mechanism exerts a torque on the compression paddle 3. The exertion of the torque transfers a mechanical pressure from the compression paddle 3 to a part of the breast B of the female patient located on an end of the breast support 2 remote from the carrier unit 1. The mechanical pressure is dimensioned in such a way that the breast B is secured. A strong pressure is not yet exerted on the breast B in this position of the compression paddle 3, however, so the breast B is not yet compressed.

FIG. 1 also illustrates a keep-free unit in the form of a displaceable plate 6 which is situated on the compression paddle 3 and with which the epidermis of a female patient can be squeezed out of the region of imaging or the radiation region.

FIG. 2 illustrates a schematic side view of the mammography compression facility 10 illustrated in FIG. 1, with the compression paddle 3 being horizontally oriented. In the phase illustrated in FIG. 2 the compression paddle 3 was displaced in the direction of the breast support 2 until the compression paddle 3 resting on the breast B is arranged in a horizontal position. In this horizontal position the compression paddle 3 is located on the breast in a planar manner and is now ready to compress the breast in a next step. For this, the compression paddle is pressed from a start position HP2′ further in the direction of the breast support 2. A carrier-side blocking device 5b blocks pivoting of the compression paddle 3 upwards and therewith swerving of the compression paddle 3 in the case of increased pressure on the breast B due to a displacement of the second stop position HP2′ downwards in the direction of the breast support is prevented and thus the compression pressure on the breast B is increased up to a predetermined value.

FIG. 3 represents a schematic plan view of a compression paddle 3 of a mammography compression facility 10 according to one exemplary embodiment with a pivoting mechanism with two hinges 5a. The compression paddle 3 has a pressure body 3a made of gauze and a frame 3b with two parallel frame elements and a rear frame element to which the pressure body 3a is fixed. A carriage guide 7 is formed on the frame on the two parallel frame elements. The compression paddle 3 also has a displaceable plate 6 which has a carriage element (not shown) and can be displaced along the carriage guide 7.

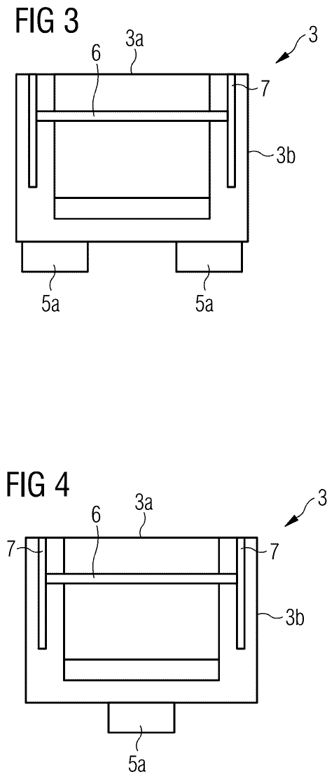

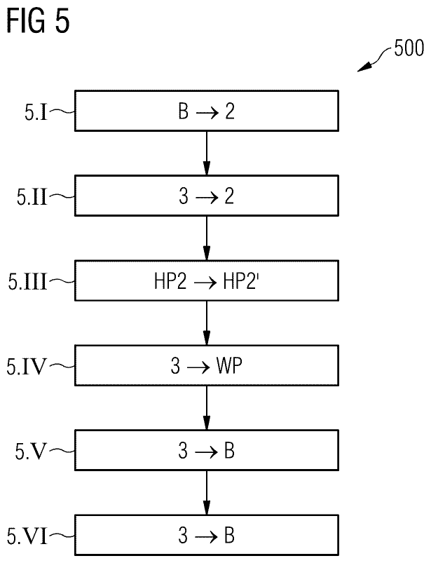

FIG. 4 represents a schematic plan view of a compression paddle 3 of a mammography compression facility 10 according to one exemplary embodiment with a pivoting mechanism with just one hinge 5a.

FIG. 5 represents a flowchart 500, which illustrates a breast compression method, which also comprises a method for automatically securing a breast B of a female patient in a compression position for a mammogram according to one exemplary embodiment of the present invention.

In step 5.I, the breast B is positioned on a breast support 2.

In step 5.II, a compression paddle 3, which is arranged at a second stop position HP2, is pivoted about a horizontal pivot axis HSA, which runs through the second stop position HP2, in the direction of the breast support 2.

In step 5.III, a height of the second stop position HP2 relative to a first stop position HP1, which is associated with the breast support 2, is changed by way of an adjusting mechanism 4.

In step 5.IV, a compression paddle 3 is pivoted back into a horizontal position WP during a reduction in the height of the second stop position HP2.

In step 5.VI, the actual compression of the breast B is carried out.

A mammogram of the breast B can then be recorded.

Finally, it should again be noted that the previously described detailed methods and apparatuses are exemplary embodiments and that the basic principle can also be varied in many ways by a person skilled in the art without departing from the field of the present invention insofar as it is defined by the claims. For the sake of completeness, it should also be noted that the use of the indefinite article “a” or “an” does not preclude the relevant features from also being present plurally. Similarly, the term “unit” does not preclude this from being composed of a plurality of components which can likewise also be spatially distributed.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

What is claimed is:

1. A mammography compression device, comprising:
a carrier unit having a first stop position and a second stop position;
a breast support fixed at the first stop position of the carrier unit and extending transversely to the carrier unit;
a compression paddle arranged at the second stop position of the carrier unit, wherein the compression paddle is configured to pivot about a horizontal pivot axis, the horizontal pivot axis running through the second stop position in a direction of the breast support;
an adjusting mechanism configured to change a spacing of the second stop position relative to the first stop position;
a pivoting mechanism at the second stop position, the pivoting mechanism configured to
pivot the compression paddle in a direction of a part of a breast of a female patient located on an end of the breast support remote from the carrier unit,
exert a torque on the compression paddle to transfer a compression pressure of the compression paddle to the part of the breast of the female patient located on the end of the breast support remote from the carrier unit, the compression pressure being sufficient to secure the breast, and
execute a pivoting-back of the compression paddle during a reduction of the spacing of the second stop position from the first stop position to implement a planar securing of the breast; and
a keep-free unit configured to keep a beam path for mapping the breast free of body tissue that is situated in a surrounding area of the breast of the female patient.

2. The mammography compression device as claimed in claim 1, wherein the compression paddle has a pressing body, wherein the pressing body includes a flexible material.

3. The mammography compression device as claimed in claim 2, wherein the compression paddle has a frame in which the pressing body is mounted.

4. The mammography compression device as claimed in claim 3, wherein the frame is formed laterally and on a side of the compression paddle facing the carrier unit.

5. The mammography compression device as claimed in claim 1, wherein the pivoting mechanism is configured to execute the pivoting-back of the compression paddle to a horizontal position of the compression paddle to implement the planar securing of the breast.

6. The mammography compression device as claimed in claim 1, wherein the pivoting mechanism comprises a friction hinge.

7. The mammography compression device as claimed in claim 1, wherein the pivoting mechanism includes a mechanical blocking device configured to limit a pivoting range of the compression paddle.

8. The mammography compression device as claimed in claim 1, wherein the keep-free unit includes a displaceable plate configured to be displaced transversely to the horizontal pivot axis up to the end of the compression paddle remote from the carrier unit, and wherein the keep-free unit is oriented transversely to the compression paddle.

9. The mammography compression device as claimed in claim 8, wherein the compression paddle includes a carriage guide running transversely to the horizontal pivot axis and the displaceable plate includes a carriage engaging in the carriage guide such that the displaceable plate is displaceable transversely to the horizontal pivot axis.

10. The mammography compression device as claimed in claim 8, further comprising:
a securing mechanism configured to secure the displaceable plate at a final position on the end of the compression paddle remote from the carrier unit.

11. The mammography compression device as claimed in claim 10, wherein the securing mechanism comprises a latching mechanism.

12. A method for automatically securing a breast of a female patient in a compression position for a mammogram, the method comprising:
positioning the breast on a breast support, the breast support being fixed at a first stop position of a carrier unit and extending transversely to the carrier unit;
pivoting a compression paddle about a horizontal pivot axis, the compression paddle being arranged at a second stop position of the carrier unit, and the horizontal pivot axis running through the second stop position in a direction of the breast support, wherein due to a pivoting mechanism at the second stop position, the compression paddle pivots in a direction of a part of a breast of a female patient located on an end of the breast support remote from the carrier unit, and a torque is exerted on the compression paddle to transfer a compression pressure of the compression paddle to the part of the breast of the female patient to secure the breast;
changing a spacing of the second stop position relative to the first stop position via an adjusting mechanism;
pivoting the compression paddle back during a reduction of the spacing of the second stop position from the first stop position to implement a planar securing of the breast; and
keeping a beam path for mapping the breast free of body tissue that is situated in a surrounding area of the breast of the female patient.

13. A breast compression method, comprising:
performing the method as claimed in claim 12; and
performing planar compression of the breast.

14. A non-transitory computer program product including a computer program loadable into a control device for actuating a mammography compression device, the computer program including program code segments to carry out the method as claimed in claim 12 when the computer program is executed in the control device.

15. A non-transitory computer-readable medium storing program segments that, when executed by a computer unit at a mammography compression device, cause the mammography compression device to perform the method as claimed in claim 12.

16. The mammography compression device as claimed in claim 4, wherein the pivoting mechanism is configured to execute the pivoting-back of the compression paddle to a horizontal position of the compression paddle to implement the planar securing of the breast.

17. The mammography compression device as claimed in claim 16, wherein the pivoting mechanism comprises a friction hinge.

18. The mammography compression device as claimed in claim 16, wherein the pivoting mechanism includes a mechanical blocking device configured to limit a pivoting range of the compression paddle.

19. The mammography compression device as claimed in claim 16, wherein the keep-free unit includes a displaceable plate configured to be displaced transversely to the horizontal pivot axis up to the end of the compression paddle remote from the carrier unit, and wherein the keep-free unit is oriented transversely to the compression paddle.

20. The mammography compression device as claimed in claim 9, further comprising:
a securing mechanism configured to secure the displaceable plate at a final position on the end of the compression paddle remote from the carrier unit.
